(19) United States
(12) Patent Application Publication
(10) Pub. No.: US 2025/0058076 A1
(43) Pub. Date: Feb. 20, 2025

(54) Sauna With Multi-Sensory Transducers

(71) Applicants: Matthew Layton Schwab, San Francisco, CA (US); David Michael Bladow, Austin, TX (US); Westin Cullen Mirner, San Francisco, CA (US); Michael Carter, Los Angeles, CA (US); Mark Edward Fleschler, San Francisco, CA (US)

(72) Inventors: Matthew Layton Schwab, San Francisco, CA (US); David Michael Bladow, Austin, TX (US); Westin Cullen Mirner, San Francisco, CA (US); Michael Carter, Los Angeles, CA (US); Mark Edward Fleschler, San Francisco, CA (US)

(21) Appl. No.: 18/671,896

(22) Filed: May 22, 2024

Related U.S. Application Data
(60) Provisional application No. 63/468,052, filed on May 22, 2023.

(51) Int. Cl. A61M 21/02; A61H 33/00; A61H 33/06; A61L 2/10; A61M 21/00; G16H 20/30

(52) U.S. Cl. CPC A61M 21/02; A61H 33/067; A61L 2/10; G16H 20/30; A61H 2033/0058; A61H 2201/0157; A61H 2201/501; A61H 2201/5082; A61L 2202/11; A61L 2202/16; A61L 2202/24; A61M 2021/0027; A61M 2021/0044; A61M 2021/0066; A61M 2205/3303; A61M 2205/368

(57) ABSTRACT

A portable sauna with multi-sensory transducers, including a heating system with a full spectrum heater and infrared panels, a sound system including an audio amplifier, speakers, and subwoofer and a lighting system including a LED controller and 16-bit LEDs. The sauna further includes an ARC server running an ARC operating system, a sensor system including an in-unit controller, a presence sensor, temperature sensor, door sensor and a power system. Still further, the sauna includes a plurality of sensors for measuring user heart rate, EEG data, respiration, cortisol levels, heat shock proteins, and heavy metals.

### RELATED APPLICATIONS

This application is a utility Patent Application claiming priority from U.S. Provisional Application No. 63/468,052 filed May 22, 2023.

### BACKGROUND

A sauna is an enclosed room, typically smaller, used by humans to experience high levels of heat in low humidity. While the concept dates back to antiquity, in more modern times, the advent of electricity enabled users to have saunas in their homes. Typically, however, the cost of installing a sauna in a home is high—due in part to the sauna heating system and the special barriers needed around the room to keep the heat from leaking into the rest of the house. Also, once installed, such home saunas could not be moved readily.

Recently, portable saunas have emerged that use infrared light for the heating system. Rather than heating the air within the portable enclosure, infrared light heats up the user inside the sauna. Additional recent developments include the use of ancillary systems such as lighting and sound systems as well as circuitry to communicate with smart mobile devices and remote servers.

Furthermore, some saunas have simple benches for users while others are known to have angled chair backs and the like.

However, these conventional saunas and related “quality of life” platforms lack a combination of transducers that provide sound, heat, and haptic output and sensors that measure user health data and generate input to the system.

As one example, in the human body, the parasympathetic state is a condition that promotes the core functions of life, including proper digestion, natural sexual arousal, easy defection, and quality sleep. The sympathetic state is the opposite, putting us in an amped-up state of “fight or flight”. The latter state hinders or impedes the parasympathetic core functions of life.

In pre-modern times, for most people, “work” was what we modern people now call “exercise”, “food” was the plants that grew from the soil and the creatures that ran, swam, or flew (foodstuffs we now call “whole foods”), and “sleep-time” was the period from sundown to sunrise. Moreover, humans are social creatures who work, play, pray, and do other activities together in the same place and time.

In those pre-modern times, and going back in time to pre-history, the parasympathetic state was the dominant or default state of humans. The “fight or flight” sympathetic state would come on only in the event of relatively rare emergencies—animal attacks, war among tribes, etc.

The industrial revolution in the 1700s is seen by some as the point when the rate of human achievements began to grow exponentially. It was in the 1700s in England when a trickle of farmers began moving to town to work in the first steam-powered factories. Some of that factory work was backbreaking (what we call “over-exercising” today). Moreover, it represented the beginning of alienation of work whereby the worker in a factory who produces a small part of a larger product never even meets any buyers of that product.

This “advancement” improved productivity of the society, but also set in motion a process of switching the default state of humans from the parasympathetic state toward sympathetic state. But it would take centuries of this process to complete the full switchover.

Between April 2020 and early 2022, it was extremely common for humans all over the world to be living, working, and playing within the four walls of their house, while sitting or lying down. They would do all their “work” using a powerful small computer connected to a blindingly fast network, and their (“manufactured”) food—much of which could last for years on their pantry shelves—would be delivered to their door via a sequence of taps on a hand-held computer more powerful than room-sized computers only 60 years before.

Through these small computers, the vast majority of human interactions took place during this time. When people did leave their house, many if not most would wear a mask hiding most of their facial features from others. When humans the world over consumed the news media during this time, the stories constantly being discussed was what some have characterized as “fear porn”. Extreme convenience is now the norm. By the present day, people all over the world are conditioned to expect that they can get most anything they want from their hand-held computer or at least one or more pills.

This extreme, agitated, socially isolated period of 2020-2022 likely represents the final and seemingly permanent switchover of the default human state to the sympathetic state, leaving the parasympathetic state as the rarer condition.

In this dystopian world, voices have emerged calling for people to engage in moderate stress to induce the increasingly rare but extremely valuable parasympathetic state. Examples of activities that cause such moderate stress include, for example, sun exposure, cold showers, intense exercise, facing our fears, and fasting. But in today's world of extreme convenience, none of the foregoing practices would be considered even remotely “convenient” by people today.

Absent from known saunas are systems or platforms for inducing the parasympathetic state in humans, in a way that is “convenient” to the modern human.

### SUMMARY

Disclosed herein is a sauna with multi-sensory transducers, including a heating system with a full spectrum heater and infrared panels; a sound system including an audio amplifier, speakers, and subwoofer; and a lighting system including a LED controller and 16-bit LEDs. In one embodiment, the sauna includes an electrical cabinet having an ARC server running an ARC operating system, a sensor system including an in-unit controller, a presence sensor, temperature sensor, and door sensor; and a power system including a power system converter and a microphone. Still further, the sauna includes a plurality of sensors for measuring user heart rate, EEG data, respiration, cortisol levels, heat shock proteins, and heavy metals.

In an embodiment, the sauna conducts experiential audiovisual synchronization wherein orchestrated interplay is created between personalized mindfulness audio content and dynamic immersive lighting to produce multimodality content programming and a guided sensory experience.

For the purpose of illustration, the discussion herein centers on one embodiment of the sauna with multi-sensory transducers. This embodiment concerns a sauna enclosure for housing a single person. However, in other embodiments, the sauna comprises a multi-person enclosure or an entire room sized space capable of housing dozens of people.

Further, in an embodiment, the sauna includes one or more technologies for directly measuring one or more of a parasympathetic state and hormesis state in the subject. These direct measurements enable the sauna to optimize the sensations for this subject, as well as demonstrate progress for the person over time.

In an embodiment, the sauna is configured to allow one or more components to be installed on the outside walls of the enclosure, enabling furniture and other useful devices to be mounted on the attaching components.

In an embodiment, the sauna includes one or more UV lamps for disinfecting the enclosure when it is empty of people.

In an embodiment, the sauna includes a reclining chair.

In an embodiment, the sauna includes a system of metal rods embedded in the floor and connected to electrical ground for ameliorating the effects of EMF on the user.

### BRIEF DESCRIPTION OF THE DRAWINGS

The disclosed embodiments have other advantages and features which will be more readily apparent from the detailed description, the appended claims, and the accompanying figures (or drawings).

FIG. 1 shows an image of the sauna of the present invention, including the major functional modules contained within, in accordance with one embodiment.

FIG. 2 shows wire diagrams of the sauna of the present invention, including (a) frontal view (b) rear view and (c) right frontal oblique view, in accordance with one embodiment.

FIG. 3 shows exploded wire diagrams of the sauna of the present invention, including (a) left frontal oblique view (b) frontal view and (c) right frontal oblique view, in accordance with one embodiment.

FIG. 4 shows a schematic diagram of the electrical connections between the panels and a plurality of modules of a sauna of the present invention, in accordance with one embodiment.

FIG. 5 shows the modules that are embedded within each block of FIG. 4, in accordance with one embodiment.

FIG. 6 shows the components of the electrical cabinet of FIG. 5, in accordance with one embodiment.

FIG. 7 shows a schematic diagram of the electrical connections between the panels and certain modules of a sauna of the present invention, including detail on the number of pins for each electrical connection, and the purpose of each pin, in accordance with one embodiment.

FIG. 8 shows components of the ARC server, in accordance with one embodiment.

FIG. 9 shows components of the ARC Service, in accordance with one embodiment.

FIG. 10 shows an exploded wire diagram of the sauna of the present invention, including a heart rate sensor embedded in a rip curl, according to an embodiment.

FIG. 11 shows the sauna of the present invention, including EMF-ameliorating features, according to an embodiment.

FIG. 12(a) shows the sauna of the present invention, including features for coupling fixtures to the outside of sauna 110, according to an embodiment.

FIG. 12(b) shows a strong framework 1220 for supporting the fixtures coupled to the outside of the sauna of the present invention, according to an embodiment.

FIG. 13(a) shows an interior view of the sauna of the present invention with a reclining chair 416, according to an embodiment.

FIG. 13(b) shows a side view of the reclining chair with an embedded reclining mechanism, according to an embodiment.

FIG. 13(c) shows an oblique view of the reclining mechanism, according to an embodiment.

### DETAILED DISCLOSURE

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### The Enclosure

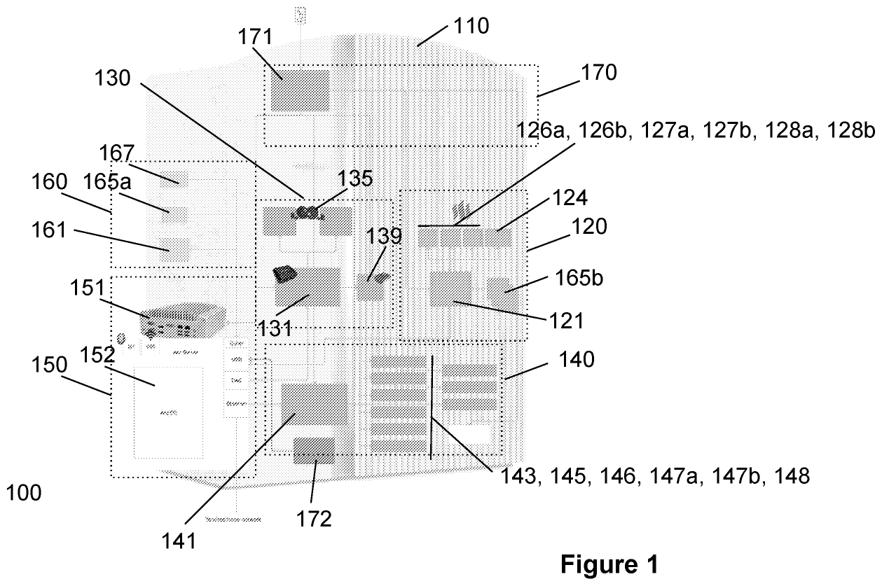

FIG. 1 shows an image of an embodiment of a sauna 110 including the major functional modules contained therein. In the displayed embodiment, these modules include the following multi-sensory transducers: heating system 120, sound system 130, and lighting system 140. These modules also include electrical cabinet 150 (which includes ARC server 151), sensor system 160, and power system 170.

Sauna 110 provides fully enclosed architectural isolation and sensory immersion and reduces visual distraction for users. Regarding eliminating external sounds, sauna 110 includes, in one or more panels, insulation known in the art that is sound dampening. Regarding eliminating external light, sauna 110 includes no windows. Moreover, in one embodiment, the outward appearance of sauna 110 is discrete and has a shallow depth footprint.

Certain components of these modules are shown in FIG. 1. In one embodiment, components of the heating system 120 include heating controller 121, full spectrum heater 124 and infrared (“IR”) panels 126a, 126b, 127a, 127b, 128a, and 128b. Sound system 130 includes audio amplifier 131, speakers 135, and subwoofer 139. Included within lighting system 140 are LED controller 141, and 16-bit pixels LEDs 143, 145, 146, 147a, 147b, and 148.

Electrical cabinet 150 includes ARC server 151, which runs ARC operating system (“ArcOS”) 152. Sensor system 160 includes controller (“IUC”) 161, presence sensor 165a, temperature sensor 165b, and door sensor 167. Power system 170 includes power system converter 171, and microphone 172.

In one embodiment, sauna 110 also includes, as part of sensor system 160, additional sensors for measuring the effects of sauna 110 on the physical health of a subject, including sensors that measure one or more of subject heart rate, EEG data, respiration, cortisol levels, heat shock proteins, and heavy metals.

As shown in FIG. 1, functional modules of sauna 110 are connected to a network that allows sauna 110 to transmit data to and from other entities, such as one or more servers, computing devices, or other saunas 110. In various embodiments, the network comprises any combination of local area and/or wide area networks, using both wired and/or wireless communication systems, such as the Internet. In some embodiments, the network transmits data over a single connection (e.g., a data component of a cellular signal, or Wi-Fi, among others), and/or over multiple connections. In some embodiments, the network uses standard communications technologies and/or protocols. For example, the network includes communication links using technologies such as Ethernet, IEEE 802.11, 4G, 5G, code division multiple access (CDMA), digital subscriber line (DSL), and the like. Data exchanged over network may be represented using any suitable format, such as hypertext markup language (HTML) or extensible markup language (XML). In some embodiments, the network includes encryption capabilities to ensure the security of user data. For example, in various embodiments, encryption technologies include secure sockets layers (SSL), transport layer security (TLS), virtual private networks (VPNs), and Internet Protocol security (IPsec), among others.

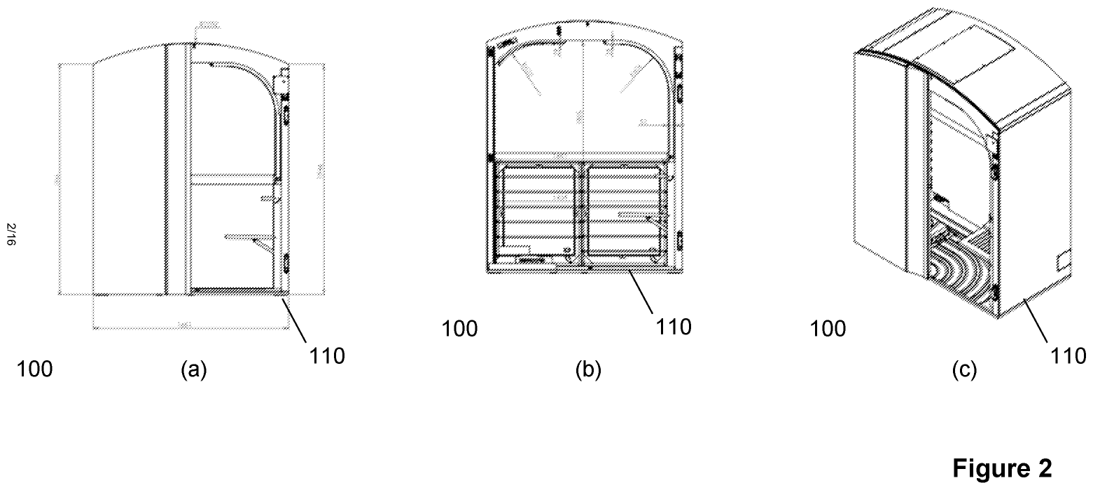

FIG. 2 shows wire diagrams of an embodiment of sauna 110, including (a) frontal view (b) rear view and (c) right frontal oblique view. In frontal view (a) of sauna 110, the door is shown as open. In rear view (b), the panel of sauna 110 is removed to reveal the interior, including infrared heating panels embedded in the lower half of this panel. In the right frontal oblique view (c), the door is also open, revealing the electrical cables along the floor (which is removed in this view).

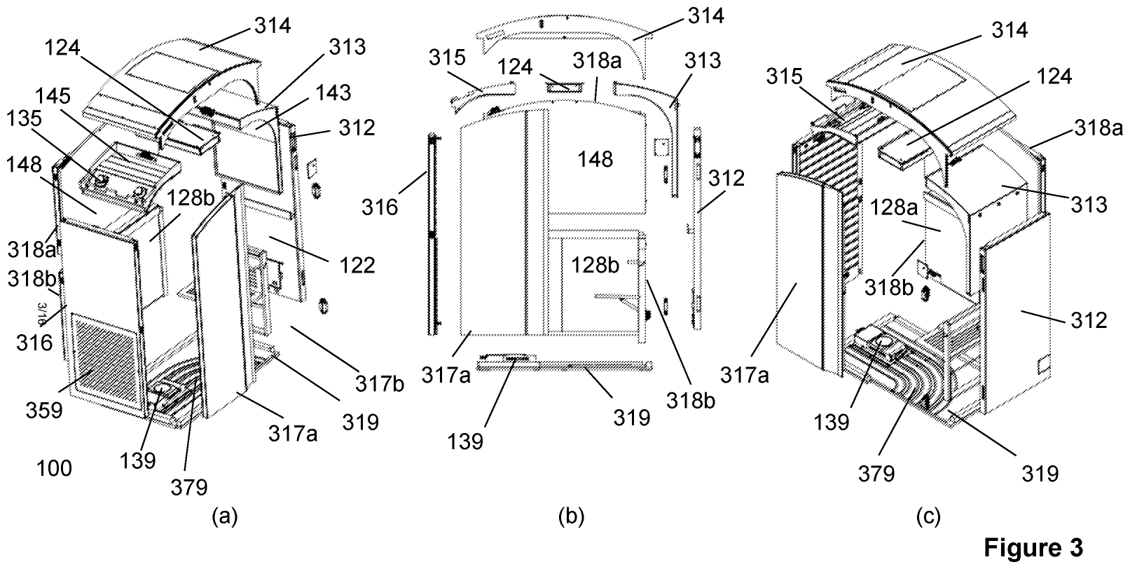

FIG. 3 shows exploded wire diagrams of an embodiment of sauna 110, including (a) left frontal oblique view (b) frontal view and (c) right frontal oblique view. This exploded view reveals that sauna 110 comprises multiple, separate panels, including front panel 312, rip curl 313, roof 314, speaker box 315, rear panel 316, right wall 317a, right door 317b (not shown), upper left panel 318a, lower left panel 318b, and floor 319.

Sauna 110 has, embedded within its panels, heating system 120, sound system 130, lighting system 140, electrical panel with control circuitry 150, sensor system 160, and electrical power system 170. FIG. 3 shows certain of the individual modules and components of these systems and where each is located with the panels of sauna 110.

Regarding heating system 120, full spectrum heater 124 (embedded in roof 314) is shown, as well as IR heating panels 122 (embedded in lower half of front panel 312), and IR heating panels 128a and 128b (embedded in the left and right side, respectively, of the lower half of left panel 318). Not shown in FIG. 3 are IR heating panels 127a embedded in right wall 317a and also 127b in right door 317b (not shown). In one embodiment, IR heating panels 122 are positioned behind fabric panels on the walls of sauna 110; full spectrum heater 124 is positioned behind a protective metal grate on the roof of sauna 110.

Sauna 110 has an optimized acoustic environment comprising a surround sound system that maximizes audio resonance and vibrational sound healing. For example, in the embodiment shown in FIG. 3, sound system 130 includes speakers 134 (embedded in speaker box 315) and subwoofer 139 (embedded in floor 319). In an embodiment, subwoofer 139 serves not only as the source of bass sound, but also the source of haptic sensations for the subject, thereby providing vibrational sound healing.

For lighting system 140, FIG. 3 shows 16-bit pixels LEDs 143 (embedded in rip curl 313), 145 (embedded in speaker box 315), and 148 (embedded in upper left panel 318a). Not shown in FIG. 3 are 16-bit pixels LEDs 147a and 147b embedded in right panel wall 317a and the door in location 317b (the door is not shown), respectively, as well as 146 (embedded in the enclosure chair (not shown)).

Regarding electrical panel 150, which is embedded in rear panel 316, FIG. 3 shows cooling fan screen 359.

For electrical power system 170, FIG. 3 shows power cables 379 (embedded in floor 319).

The sensor system is not shown in FIG. 3. However, in the embodiment of FIG. 3, certain sensors are embedded in speaker box 315 and in right panel wall 317a.

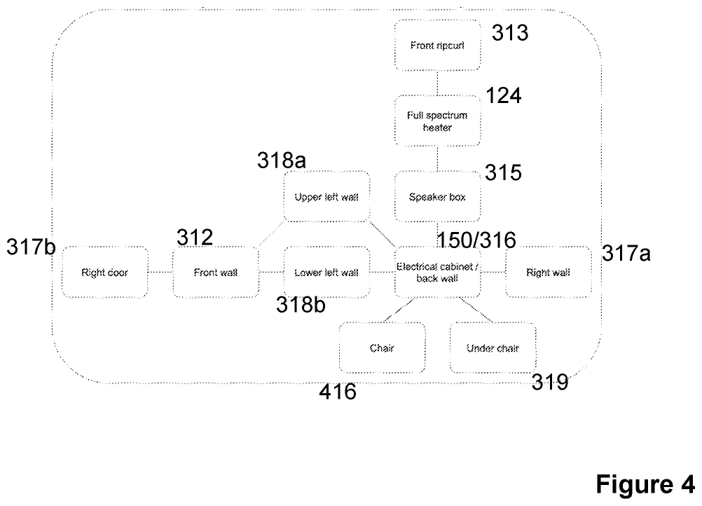

FIG. 4 shows a block diagram of the electrical connections between the panels and certain modules of sauna 110, according to an embodiment. Specifically, FIG. 4 shows rip curl 313 connected to full spectrum heater 124, which is connected to speaker box 315, which is connected to electrical cabinet 150 embedded in back wall 316, which is connected to right wall 317a, floor 319, chair 416, upper left wall 318a and lower left wall 318b. In FIG. 4, each of lower left wall 318a and upper left wall 318b is connected to front wall 312, which is connected to right door 317b. The electrical connections are bi-directional.

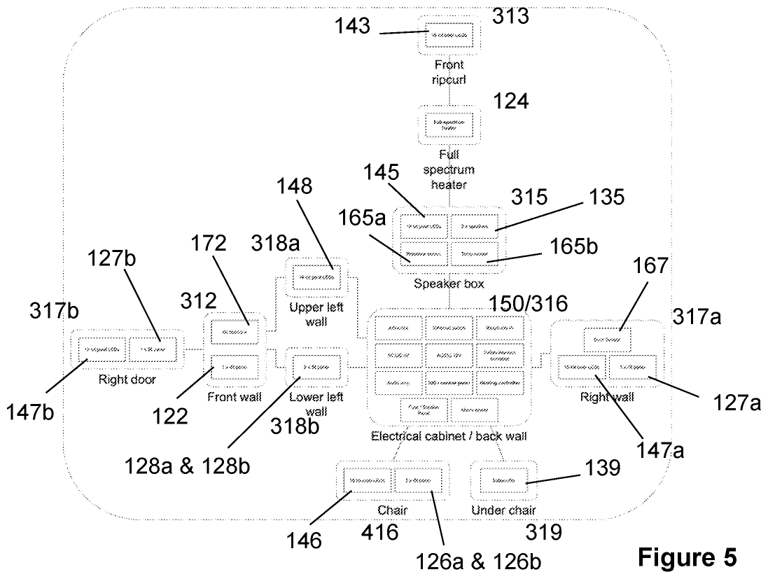

FIG. 5 shows the modules and components that are embedded within each block of FIG. 4. According to an embodiment of FIG. 5:
rip curl 313 embeds 16-bit pixel LEDs 143;
speaker box 315 embeds not only speakers 135, but also 16-bit pixel LEDs 145, presence sensor 165a, and temperature sensor 165b;
right wall 317a embeds 16-bit pixel LEDs 147a, IR heating panel 127a, and door sensor 167;
upper left wall 318a embeds 16-bit pixel LEDs 148 and lower left wall 318b embeds IR heating panels 128a and 128b;
right door 317b embeds 16-bit pixel LEDs 147b, and IR heating panel 127b;
chair 416 embeds 16-bit pixel LEDs 146 and IR heating panels 126a and 126b; and
floor 319 embeds subwoofer 139.

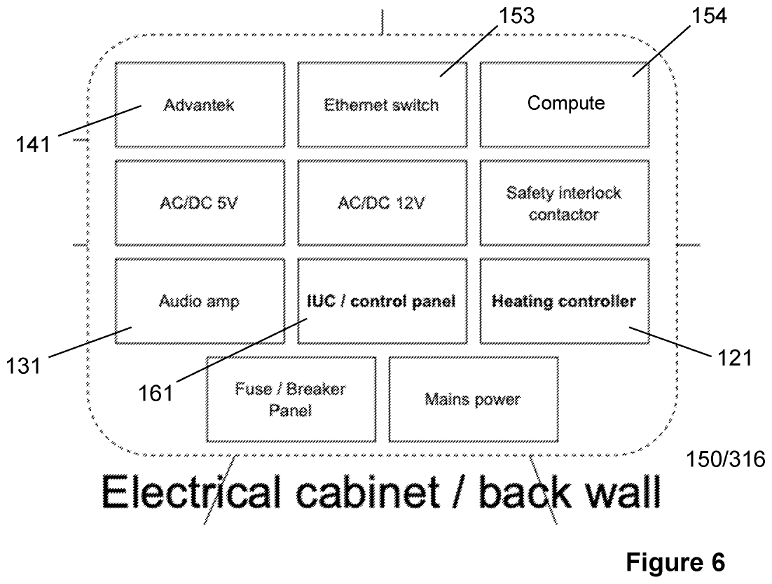

FIG. 6 shows one embodiment of the components that are embedded within electrical cabinet 150, which, in turn, is embedded within back wall 316. In this embodiment of sauna 110, electrical cabinet 150 contains heating controller 121 of heating system 120, audio amplifier 131 of sound system 130, IUC 161 of sensor system 160, LED controller 141 of lighting system 140, Ethernet switch 153 and Compute 154 of ARC server 151. In various embodiments, compute 154 (also referred to herein as “compute module 154”) is a computing platform such as Raspberry Pi, Nitrogen 8MP, or such platforms known in the art. The remaining components of electrical cabinet 150—AC/DC 5V, AC/DC 12V, Safety interlock contactor, Fuse/breaker panel, and Mains power are part of power system 170.

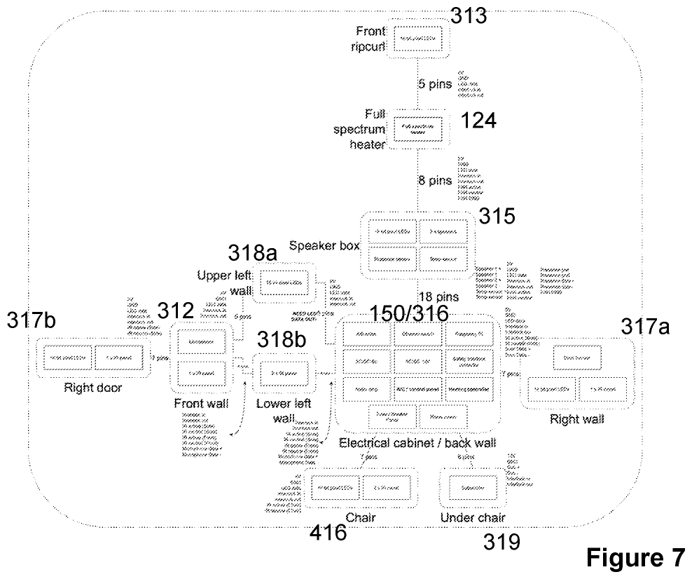

FIG. 7 shows a schematic diagram of the electrical connections between the panels and certain modules of sauna 110, including detail on the number of pins for each electrical connection, and the purpose of each pin, according to an embodiment. For example, the electrical connection between rip curl 313 and full spectrum heater 124 uses 5 pins, their functions being 5V, GND, LED data, Interlock in, and Interlock out.

FIGS. 1 through 7 show a physical and electrical layout and configuration of an embodiment of sauna 110. Other embodiments of sauna 110 use different layouts and configurations. For example, in the particular embodiment discussed thus far, the only component embedded in roof 314 is spectrum heater 124. But in other embodiments, other components are embedded in roof 314, such as one or more of a set of IR panels, one or more sensors, and one or more components of sound system 130. Further, other embodiments make use of other multi-sensory transducers known in the art for creating lighting, sound, heating, haptics, aromas, and other sensations.

### ARC Server

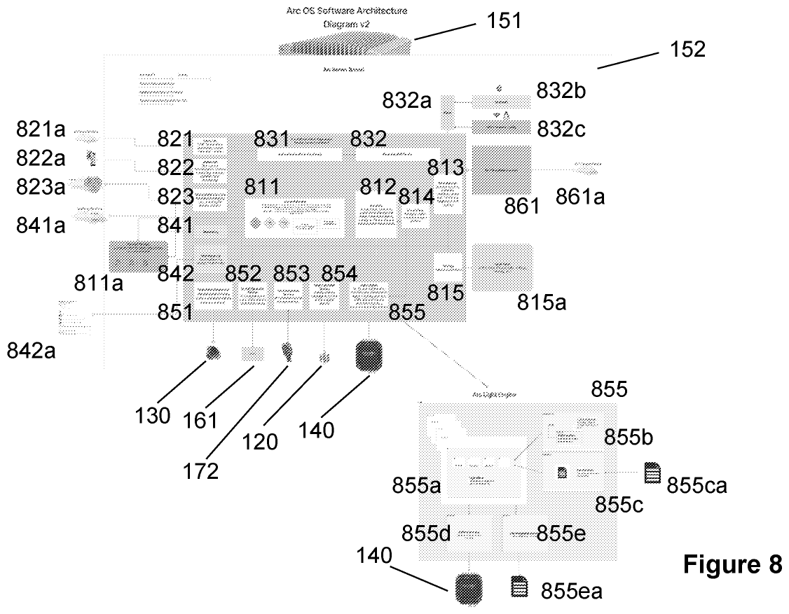

FIG. 8 shows components of ARC server 151 according to an embodiment. This server runs ArcOS 152, which acts as a standalone, always running embedded software application that lives inside compute module 154 as the control mechanism and brain for the hardware subsystems within sauna 110. Additionally, this software interfaces with the Arc mobile application 822a, over the air (OTA) application and operating system update mechanism 861, APls 821 that manage content delivery/streaming, product activation, and remote operations.

The software architecture of ArcOS 152 centers around ArcState 812, which is a state object that stores attributes that can be modified by various inputs (e.g., the mobile app 822a, ARC services 821a, or asynchronous running processes in the Arc OS application itself), which then spawns events that inform various components and subsystems on what to do.

For instance, when ArcState 812 detects that the “playback state” has changed to “play”, audio, lighting, the In Unit Controller 161 responds accordingly and renders the desired content to respond.

### States that Arcos 152 Listens to and Stores

Playback state (e.g., stop, play, pause, restart/rewind)
Playback/Session time
Set Content (class or session content)
Set Mood (looping audio/light preset)
Interior State (session, post-session, lobby, off).
Sensor State (door sensor, presence sensor)
Temperature (current temperature in the unit)
Target Temperature (desired temperature to heat up to)
Full Spectrum level (0-5 levels of power to full spectrum light fixture)
Current User (user currently using Arc)
Main Volume
Brightness
Mood Volume
Power (soft on/off or standby mode)

Core Runtime 813 provides an event loop that listens to state changes both internally and via sensors that perform actions on various subsystems like lighting, audio, heat, and online interactions.

Settings 815 provides a means of storing various settings, such as volume level, brightness and other preferences in between subject uses of enclosure 110. These values are stored within Local Store 815a.

Authentication & Identity (User) 831 manages the settings for the active subject of enclosure 110 and authenticates the validity of said subject.

Monitoring Service 841 provides a telemetry service that monitors usage (when connected online) as well as any issues that might occur during a subject's use of enclosure 110 that can be detected via software.

Web Admin UI 842 provides a web interface 842a that is accessible to the developers of enclosure 110 for testing and demonstration purposes.

Lighting in sauna 110 utilizes 16-bit LED pixel control technology, which allows for an immersive lighting experience in low level light that feels organic and remains gentle on the eyes, even when they are adapted to the dark. This is executed inside a 16-bit lighting engine that builds around the concept of a Pixel using 16-bit numbers rather than 8-bit in more traditional LED systems.

Moreover, the present disclosure recites various modes of expression to create light content via Arc Light Engine 855. It provides the concept of Light Action 855b, which produces light over a range of pixels that can be shaped linearly, using waveforms like a sine wave, and Gaussian curves. ArcOS 152 layers and combines these shapes to create elaborate and complex lighting experiences.

These actions house an array of values that are then placed into Light Mixer 855a that maps values to OMX channel 855d and splits the most significant and least significant bit from 16-bit value so it can be transmitted as two 8-bit values for established protocols like ArtNet. In one embodiment, the lighting engine records and imports lighting data and plays back content from a file in time to music.

ArcTimer 814 is a clock or timer that calls a function periodically (e.g., every 25 milliseconds (i.e. 40 hz)) to compute and render lighting for the entire system. In one embodiment, this is done by calling a “sum” function in Light Mixer 855a, which adds all of the various actions calculated values for each channel into a single level of brightness to send to lighting controller 140, producing a “frame”. Light Mixer 855a either writes this frame to file 855ea via File Rendering Service 855e, or outputs it to controller 140 via ArtNet 855d.

Audio Mixer/Sequencer 851 layers and transitions system sounds and music with selected content. This is managed via a software “mixer”.

In one embodiment, Arc State 812 defaults to a “Lobby State” for most passive use. This state is a warm, comforting state where the light is soft white and a looped, immersive, and passive audio soundtrack plays. When playing a mood or content session, Arc State 812 then “transitions” this state to create a smooth and seamless shift in color and sound for the user. When a session is completed or the user stops the session early, Arc State 812 seamlessly transitions back to the lobby state, but with dimmer lights. In one embodiment, a pre and post session sound is played during this transition.

Content Storage 811a stores content as an audio track, lighting file and metadata. This bundle is cached locally in ARC Server 151, and ArcOS 152 reads an index file that tells the software where various content assets are. A mechanism to add/remove and update content works in concert with the cloud content service 823 to download (from, for example, AWS S3 832a) and store them on the device, generally while not in use. This is controlled via mobile app 822a, or internally via admin/operation 811.

Bluetooth 832b is a service that manages syncing mobile app 822a or an audio source via Bluetooth.

Wifi & Network config 832c manages the internet connections for enclosure 110 through a local wireless network or wired ethernet connection.

Content takes the form of Moods, which act as presets and loop lighting functions and a backing immersive nature soundscape, and can run indefinitely as a “Class,” which guides the user through an exercise via the audio track and visual cues and a custom lighting track, an immersive audio experience in which music and lighting alone guide the experience, or a “low fi” class that utilizes an existing lighting preset or mood to back the guided audio content passively.

ArcOS 152 is also utilized initially in the creation of lighting content. This is managed by creating mappings of Light Actions, which shape light, color, and progression over time to Midi events.

This allows for the easy utilizing of existing digital audio workstation software to conveniently program lighting to audio content. When a mapped Midi note is detected, it allows for a user (e.g., a lighting designer or creative) to build complex, immersive 16-bit light sequences.

Mobile interactions (via mobile API 822) and remote interactions via cloud content services 823 call APls via gRPC, allowing for symmetry and consistency across multiple platforms.

In an embodiment, one or more of the major functional modules contained within sauna 110 include firmware. For one or more of these instances of firmware, the firmware is updatable via OTA update mechanism 861, which obtains the updates from OTA Update Service 861a.

Power system 170 includes independent firmware that is updatable.

IUC 161 also includes independent firmware that is updatable. This firmware acts as a “dumb” controller to write data to a small OLEO display, detect button presses and turns of a rotary encoder knob. The UI and overall logic handling the interaction events (i.e. subject presses button A) is processed inside ArcOS 152 via Arc IUC Controller Service 852. The IUC firmware manages routing of the state of buttons, keypresses, and screen via a single serial connection to compute module 154. ArcOS 152 includes a service that listens for interactions with IUC 161 via Controller Service 852 and produces updates to ArcState 182 that drive various output functions discussed earlier.

Heating system 120 attempts to maintain a targeted temperature, using similar mechanisms that a home thermostat would: heat until target temperature is met, when temperature falls below a tolerance, heat until target is met, and then repeat. ArcOS 152 communicates with Heat Controller Service 854 that makes direct function calls to the hardware heating controller of system 120. Essential functions include turning on and off full spectrum heater 124 and infrared (“IR”) panels 126a, 126b, 127a, 127b, 128a, and 128b, while also reading temperature sensor 165b.

Lighting system 140 implements ArtNet 855d client, which ingests 8-bit dmx values for each channel (every pixel represents 8 channels: red, green, blue and white×2 for 16-bit brightness) via UDP and routes the values to the appropriate LED strips and pixels, creating light.

Regarding sound system 130, ArcOS 152 mixes its audio down to a single output that is routed to a DAC that is connected via the compute 154 1/0. The DAC converts the digital audio signals to analog and are then sent to the speakers for playback in enclosure 110.

For sensor system 160, door sensor 167 and presence sensor 165a inform ArcOS 152 whether door 317b is open or not and a person is inside sauna 110 or not, respectively. The firmware of Sensor system 160 outputs a binary value, updating ArcState 182.

### Arc Services

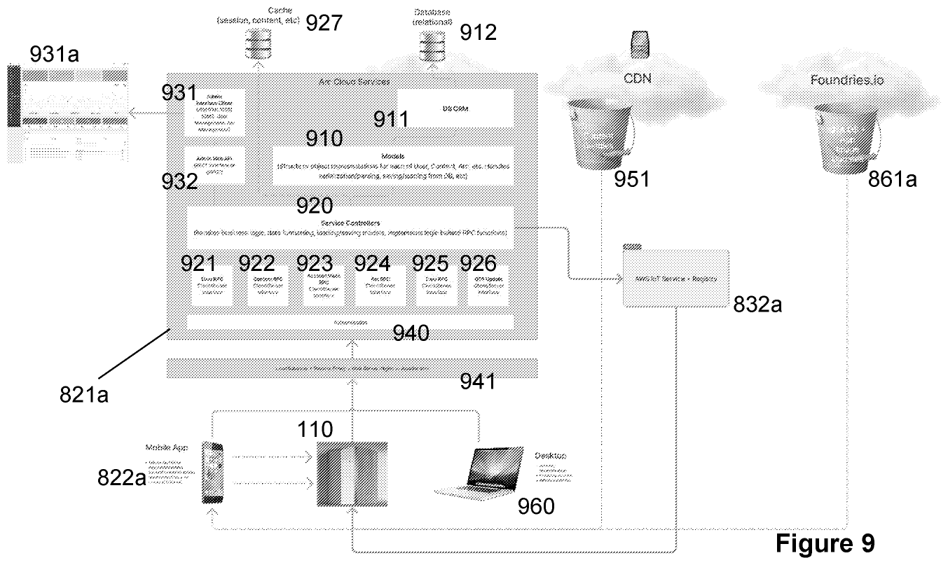

FIG. 9 shows components of ARC services 821a according to an embodiment. ARC services 821a handles the management of three primary entities: Subjects, Saunas, and Content. Data models for these entities are defined by Model Service 910. All of these are represented in database 912 via DB ORM 911. In various embodiments, data corresponding to subjects (e.g., identifying information and settings associated with users of sauna 110), saunas 110, and content is stored and processed at one or more of ARC server 151, mobile app 822a, and/or ARC services 821a.

### Subjects

Entities are created when a subject either purchases sauna 110 via Desktop 960 or when the subject logs into mobile app 822a for the first time when creating an account. Subjects access (via Front End 941), authenticate (via Authentication Service 940), and view identifying information and settings and usage information via ARC services 821a.

Subject data is ingested via ARC services 821a, primarily via use of ARC server 151, but also through the usage of mobile app 822a as well.

### Sauna

ARC services 821a creates an entity for each sauna 110 upon its being manufactured and flashing its compute 154 for the first time. As part of creating this entity, ARC services 821a generates an identity for the sauna 110.

Each sauna 110 interacts with ARC services 821a when accessing and updating its stored content, and uploading information and data provided by the subject, such as interactions with the sauna, usage of the microphone and any other sensors.

### Content

ARC services 821a is responsible for hosting and managing content library 951, which is available to sauna 110 and the subject. Content library 951 hosts high resolution audio and prerecorded lighting data to play back on sauna 110 in addition to metadata related to any additional output mechanism (scent or haptics, for example), and either downloads the content for future playback onto sauna 110 or live streams it. In an embodiment, two-way interactions could occur during a session and thus the server facilitates this interaction.

In one embodiment, metadata is hosted by the server directly (via database) but the content assets are referenced via a CMS system or secure asset host.

### Service Controllers

Service Controllers 920 provide the business logic layer of ARC services 821a, handling data conversion (for example, creating new Content or new entities in the database) and determining the content or recommendations that are made for subjects, which communicate with RPCs 921-925 and AWS S3 832a.

Cache 927 provides a caching layer such as memcache (a RAM/memory-based cache service), which enables faster responses to commonly made requests to ARC services 821.

RPCs 921-925 comprise APls for various interactions with ARC services 821a; mobile app 822a, enclosure 110, and even e-commerce integrations will all access these APls.

### Admin Management

The server also hosts a secure management console for non-engineers to engage with the system. A UI 931a is available for administrators to oversee, via Admin Interface Client 931 and Admin Web API 932, usage information and functionality of the Arcs in the field that are actively connected.

Admins also manage the content available to the user base, by adding or editing the content library and its metadata, scaling the growth of the library.

### Mobile App

One of the primary ways of interacting with the server, from the perspective of the user, is via the mobile app 822a. Via the mobile app 822a, the user may browse content and even control and monitor the state of the Arc via the server, so they know if it's in use even when not at home, and be able to preview and queue content as well as usage history and personal health progress via usage.

### Third party integrations

The service is configured to integrate with third parties, including some CMS and e-commerce integrations. In one embodiment, the service is also configured to integrate with OTA (over the air) update service 926 (which obtains the updates from OTA Update Service 861a) or support hosting one to enable the ability for remote updates to the Arc's firmware.

### Microphone and Self-Coaching

In one embodiment, sauna 110 is configured to induce a parasympathetic state in a user in part via microphone 172. Alternatively, IUC 161 or mobile app 822a are used to collect input from the user.

In an embodiment, a recording function is cued by a timecode audio file such as a .yml file. For example, at 03:45, ARC server 151, via Arc Microphone Service 853, activates microphone 172 and makes a recording based on an audio guided prompt in which the subject's response time is fixed for 30 seconds from 03:45-04:15. Other fixed intervals can be used in other embodiments. The same holds true for other intervals discussed in the examples below.

In an embodiment, the recording function is cued by an automatic vocal sensor, thereby providing dynamic responsiveness, as known in the art (e.g. Siri® on an iPhone®). In this case, ARC server 151 automatically begins recording when the subject begins speaking, and the subject's response time is dynamic and responsive. For example, ARC server 151 allows 5 seconds for the subject to initiate speaking in response to an audio guided prompt, plus 5 seconds of post-speaking silence to trigger the next step of audio guidance.

Per the foregoing, microphone 172 input to ARC server 151 is isolated from audio playback via an override mechanism that ensures microphone 172 picks up only the subject's vocal audio and not the audio guided content that is playing within sauna 110.

In an embodiment, ARC server 151 employs visual lighting cues to indicate ‘start’ and ‘stop’ instructions for microphone 172 recording function. For example, the audio guidance explains to the subject that a green light will appear when microphone 172 is actively recording and instructs the subject to speak their response during this time.

In an embodiment, this lighting instruction is cued by fixed time codes, for example, at 03:45, 16-bit pixels LEDs 143 undulate green for 30 seconds, to indicate active recording.

In an embodiment, this lighting instruction is cued by an automatic vocal sensor (dynamic responsiveness), thereby providing dynamic responsiveness, as known in the art (e.g. Siri® on an iPhone®). For example, when ARC server 151 senses the subject speaking, audio guidance pauses and 16-bit pixels LEDs 143 undulate green for the duration of vocal input to indicate active recording, plus 5 additional seconds of post-speaking silence to trigger the next step of audio guidance.

In an embodiment, ARC server 151 transcribes its recordings of the subject's intention-setting and post-ritual reflections. Sauna 110 employs transcription software known in art to create these documentations of in-session vocal recordings. Following transcription, ARC server 151 uses word cloud and heat map software to organize the text via word clouds and topic-based heat maps to assemble an overall picture of the subject's personal growth goals.

In an embodiment, the disclosed components of sauna 110 analyze the subject's voice recordings, transcriptions, and word cloud/heat mapping, to continually improve the subject's experience. For example, at the start of the subject's use of sauna 110, ARC server 151 provides limited, predetermined combinations of content programming according to the most common needed states for new subjects, for example Anxiety, Stress, Exhaustion/Sleep, Performance.

Then, ARC server 151 assigns programming (e.g., predetermined playlists) to the subject as a prescription for their personal growth goals and serves this programming to them as a Collection or Program via mobile app 822a. For example, based on the word clouds that ARC server 151 created during the subject's intention setting recordings, ARC server 151 determines that the subject is interested in Performance, and thus provides a Program made for the subject that will guide them through their growth journey.

In an embodiment, the same vocal input collection, transcription, and organization process described above is used to assemble a picture of the subject's post-ritual reflections (e.g. end-of-session moods). ARC server 151 then delivers this picture to the subject via one or more of mobile app 822a, email, and IUC 161 to convey progress throughout specific touch points and milestones in their program journey. In an example picture, the message is: “Congratulations! You've just completed 4 weeks of the Performance program. Here's a look at the progress you've made”. The message in the example is accompanied by visual side-by side comparisons of word cloud heat maps, e.g., generated using existing software, that depict their original need state versus end-of-session blissful feelings.

In an embodiment, ARC server 151 or ARC services 821a adapts the foregoing messages as templates that are auto-generated, auto-filled, and scheduled for automatic dispersal.

### Inducing the Parasympathetic State

Scientific studies have demonstrated the efficacy of therapies based on light, sound, smell, haptic, heat and other modalities for improving the health of people. But such studies, of necessity, blur the distinctions between individuals to show this positive effect.

But in the area of human health, direct measures on an individual are much more accurate and useful than studies for determining which kinds of and what combinations of therapies based on light, sound, smell, heat, haptic and other modalities are effective for that particular individual.

The sauna 110 disclosed herein provides combinations of multi-sensory transducer sensations optimized for each subject. It includes three embodiments for doing so, each successfully deeper, more accurate, and more efficient.

### Content for Any Subject

In an embodiment, sauna 110 conducts experiential audiovisual synchronization wherein orchestrated interplay is created between personalized mindfulness audio content and dynamic immersive lighting to produce multimodality content programming and a guided sensory experience.

Content for an experience is expressed via one or more layered audio tracks, a lighting track or algorithm, and additional metadata to support other transducers, such as a scent module. This data is stored on ARC server 151 or in ARC services 821a and played back directly from the former or streamed remotely from the latter. The subject queues this content, using either mobile app 822a at that moment, or pre-scheduling the content earlier. The content is either suggested as part of a class or series of classes or chosen ad hoc by the subject.

Once the content is queued, if it is not live and streaming from ARC services 821a, when the subject enters sauna 110, the user initiates playback by pressing the “play” button on IUC 161 or mobile app 822a. Content, once playback has initiated, seamlessly transitions from the “ready state” or “lobby state” of lighting and audio, which is a looping state of light and audio that invites the subject as they enter and situate themselves in sauna 110.

Lighting is seamlessly transitioned into the colors and brightness of the content lighting track or algorithm and the audio crossfades into the content audio once initiated. A “splash” sound dynamically plays on top. The control software running on ARC server 151 orchestrates this synchronization by dynamically “mixing” audio and lighting tracks upon receiving the request to begin playback.

A typical content experience includes lighting, audio and other sensory outputs working in concert with each other. In various embodiments, these sensory outputs are either pre-orchestrated or involve live dynamic interaction. For example, in a breathwork session, lighting includes visual cues that guide the user into timing their breathing. For instance, when the guide says to breathe in, the lighting reacts by becoming brighter in a location along the beltline and/or changing colors/texture/patterns as a visual cue in sync with the audio.

For meditation, lighting reacts similarly and is in sync with instructions by a guide for the purposes of facilitating a visual/emotional response. For example, when the guide asks the subject to feel their heartbeat, a soft red light that brightens in a rhythm that emulates a heartbeat. In an embodiment, the subject is wearing a heath device that records the subject's actual heartbeat, which data is shared with ARC server 151, and, in this case, the rhythm matches the subject's actual heartbeat.

In an embodiment, when a guide asks the user to think of someone they love, a pattern of purple and red light is faded, accentuating the emotional response of the user.

For pre-recorded content, lighting and audio is pre-rendered and played back in sync similar to video/audio playing back in sync. ARC server 151 includes audio/lighting services inside the controller application that manages playback and rendering of both, and that listen to and execute events such as “Play” “Pause” “FF/Rewind”. Brightness and volume are also dynamically controlled by the subject and can also be seen as events both to which the controller application responds simultaneously.

If a content experience is live or two-way (i.e. the subject is engaged in a live interaction with a human guide), the subject uses microphone 172 as a means of dynamic interaction. ARC server 151 records and streams the subject's voice to ARC services 821a and is received and heard by the live human guide using ARC services 821a as the intermediary. In an embodiment described earlier, the subject's voice is used as sensory input to live and dynamic lighting that is layered on top of a pre-rendered track or live algorithm, creating a synesthetic interaction.

Once the content experience ends, either automatically reaching its conclusion, or with the user manually stopping it, the audio and lighting transition back to a dimmer and quieter version of the ready/lobby state, which acts as a cue to the user to either queue up different content or exit sauna 110. This state lasts into perpetuity until the subject leaves sauna 110. Presence sensor 165a and door sensor 167 inform the controller application that the subject has left, and the application initiates lighting and audio to shut down and seamlessly fade out.

### Content for This Subject Based on Their General Third Demographic Data

In an embodiment, the subject is invited to share general demographic information about themselves, including one or more of age, sex, height, and weight. With this general data collected from a plurality of subjects, data science techniques are used to determine the most effective combinations of therapies for each subject depending on their shared general data.

### Content for This Subject Based on Their Shared Third Party Data

In an embodiment, the software platform associated with sauna 110 determines the most effective combinations of therapies, including the suggested duration and recommended frequency of sessions, based on deeper data about the subject that can change over time and between sessions. Such changing data includes one or more of the degree of “busyness”, and heart disease, sleep problems, athletic performance, job type, temperament, and other such factors. In this embodiment, the subject chooses to share the foregoing data with the platform, via one or more of mobile app 822a, sauna 110, and ARC services 821a.

Regarding busyness, upon request, the subject has shared one or more indices of busyness, including, for example, smartphone screen time, online calendar, phone call frequency and duration, numbers of emails sent, etc.

The more screen time, the more crowded the calendar, and the higher the frequency and/or duration of phone calls, the greater the number of emails, the greater the busyness of the subject. The platform weighs this data shared by the subject so that the more recent dates are more important than earlier dates.

In an embodiment, if the platform determines that the user has been spending an excessive time on their smartphone or has a full schedule of appointments over the previous 24-48 hours, it automatically adjusts the programming tailored to the user's current level of stress and busyness. In this example, the platform recommends a specific breathwork technique that is known to be particularly effective for reducing stress and anxiety, such as 4-7-8 breathing or alternate nostril breathing. Additionally, or alternatively, the temperature of sauna 110 is increased to enhance the relaxation effects of the session.

The foregoing example applies equally to shared data on the subject's health profile, if such data indicates greater stress.

Regarding subject data such as heart disease, sleep problems, athletic performance, job type, temperament, and other such factors, data science techniques are used to determine the most effective combinations of therapies for each subject depending on this shared deeper, changing, data.

### Content for this Subject Based on Directly Reading their Parasympathetic State

In an embodiment, sauna 110 includes one or more sensors for directly measuring the parasympathetic state of the subject. The present disclosure emphasizes convenience that the modern human expects, and direct measures taken from an individual's body are much more convenient for the subject to understand than asking that subject to read and parse or blindly accept the generalized results of scientific studies, or even engage in the previous two embodiments for optimizing the experience.

In this embodiment, such sensor measurements are taken at the beginning of the session, at the end of it, at both the beginning and the ending, and/or periodically during the session (for example, every minute or multiple minutes).

In an embodiment, the results of these direct measures are presented to the individual during the session, after the session, or in both times. They are presented in one or more of mobile app 822a, a screen within sauna 110, and a screen outside sauna 110.

One primary purpose of these sensors is to determine whether the subject's body is trending, over multiple sessions, toward the intended parasympathetic state of reduced stress and increased relaxation, or toward the unintended, opposite, sympathetic state of increased stress and agitation.

As noted earlier, the multi-sensory transducers of sauna 110 produce hormesis—a moderate level of stress—in people. Accordingly, at the beginning of a platform session, it is expected that indicators of stress in the subject will increase.

But also, as noted, the purpose of producing hormesis is to develop resilience in the subject across multiple sessions within sauna 110. This increased resilience should produce a decreasing level of stress across the timeframe of a single session, and across multiple sessions. This is why sauna 110 records multiple measurements over the period of each session.

The pattern of these measurements over the session, and between sessions, can indicate lowering stress (and thus a higher parasympathetic state), which is a desirable pattern.

Alternatively, the pattern can indicate the opposite: greater stress, and thus a greater sympathetic state, which is an undesirable pattern.

The human processes that these sensors measure include one or more of cortisol, heart rate, voice intonation, and brain waves.

Cortisol is a steroid hormone the level of which increases in response to greater stress, and decreases with resilience, and thus lowers stress. Thus, the higher the cortisol level, the lower the parasympathetic state; the lower the cortisol level, the higher the parasympathetic state.

In an embodiment, sauna 110 includes one or more sensors such as those developed by CalTech and Stanford for continuous measuring of cortisol levels in human sweat. These sensors are flexible, wearable, and protected from corrosion by the human sweat that they measure. In various embodiments, such a sensor is placed on an armrest on chair 416; in a bar or other shape that the subject holds gently in one hand throughout the session; or within an article of clothing (for example, an arm or wrist band, a headband, a hat, etc.) worn by the subject that maintains contact with the skin.

Certain heart rate patterns are known in the art to indicate a parasympathetic state. One such pattern, known as the “Valsalva ratio”, is defined as the maximum heart rate during the period of interest divided by the lowest heart rate obtained within 30 seconds of the peak heart rate. In various embodiments, the period of interest in a session is the entire session or a subset (e.g. during a single Breathwork segment).

Another heart rate pattern known in the art for indicating a parasympathetic state is heart rate variability (HRV). This particular HRV pattern that shows a parasympathetic state is the subject of debate in the current art as of 2022. Some of these HRV patterns used in the art are the ratio of HF to LF, rMSDD, Spectral analysis, and PAI/SAi.

In one embodiment, sauna 110 uses a heart rate sensor to calculate one or more of the HRV patterns and the Valsalva ratio. In an embodiment, sauna 110 includes one or more of a remote infrared sensor, and an RGB camera. The first measures heart rate directly; the second, via measuring color changes in the skin and applying known functions to compute heart rate.

Still other methods known in the art combine both kinds of remote sensors to compute heart rate.

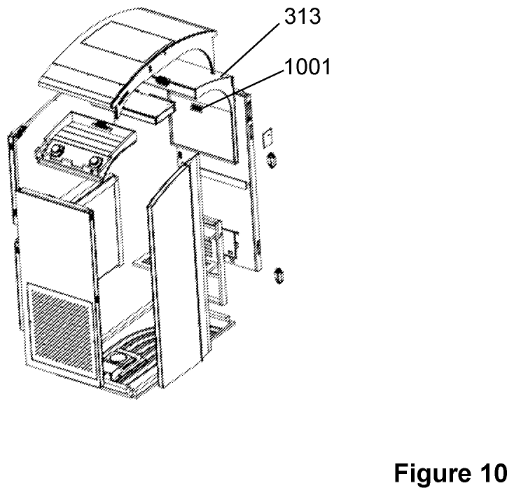

Sauna 110 positions these sensors remote from the subject, for example, in rip curl 313, as shown in FIG. 10 with sensor 1001.

In an embodiment, the subject wears a device during the session that measures heart rate—such as an Apple Watch®, a FitBit® Watch, an Oura® Ring, etc.—and opts to share that data with sauna 110. Sauna 110 identifies the sets of time series in that shared data that correspond to sessions of the subject in sauna 110. With those sets of time series data, sauna 110 extracts the heart rate data, and computes one or more of the heart rate patterns described above.

In an embodiment, sauna 110 uses microphone 172 as a sensor for indicating a parasympathetic state. In one usage, microphone 172 is used to collect “self-reports” from the subject. That is, the sound system 130 queries the subject on his or her state of relaxation, and records the answers spoken into microphone 172.

In an embodiment, ARC Server 151 uses microphone 172 to implement a dynamic self-coaching system whereby sound system 130 queries the subject to log personal thoughts and reflections for self-improvement and self-awareness. Microphone 172 captures the responses and synthesizes them to deliver a programming prescription to address the subject's desired personal growth outcomes, as described earlier.

In an embodiment, ARC Server 151 uses microphone 172 to measure voice intonation. Voice intonation refers to the emotional quality of voice. Higher versus lower levels of stress cause differences in voice intonation. In an embodiment, during a session, sound system 130 queries the subject, once or multiple times, for a more complex answer (i.e. not simple “yes” or “no”).

Sound system 130 records these detailed answers and applies known techniques to measure voice intonation. In one embodiment, measurements taken over multiple sessions are compared and used to evaluate the subject's state of stress.

In an embodiment, sauna 110 includes a flexible skull cap that includes sensors for measuring EEG data arising from brain waves on the surface of the head. In an embodiment, the cap embeds other flexible sensors, including, for example, a flexible cortisol sensor as discussed earlier.

At the start of one or more sessions, the subject is asked to wear the cap during the entire session. One purpose of the EEG sensors is to measure activity levels in the default mode network (“DMN”) of the brain. Known techniques are used to compute the level of activity in the DMN via EEG.

The DMN network comprises multiple areas of the brain. It is known in the art that during states of effective meditation, there is reduced activity in the OMN. Sauna 110 periodically or continually measures activity levels in the DMN via the EEG skull cap. With this collected, sauna 110 is able to directly measure the effectiveness of the subject's meditation.

### Closing the Loop

In an embodiment, shortly after a session has completed, the subject is asked to rate the session. This request is delivered via one or more of IUC 161, mobile app 822a, and sound system 130. The subject is invited to enter her rating via one or more of the following means: typing on IUC 161 or mobile app 822a and speaking. For the latter, microphone 172 is used to capture the subject's rating, and comprehension of the same is accomplished as discussed earlier in the context of the microphone and self-coaching.

### This Rating Concerns One or More of the Entire Session, and Individual Segments of the Session

In an embodiment, shortly after a session has completed, the rating is automatically computed based on the sensor data gathered from the subject during the session (as discussed earlier). A greater indication of resilience detected within the subject results in a higher automatic rating.

Sauna 110 uses one or more of these ratings—provided by the subject and computed automatically—to determine an optimal combination of light, sound, smell, heat, and haptic sensations for inducing an ideal degree of hormesis in that subject as measured by these sensors.

In an embodiment, sauna 110 iterates toward this optimal combination across multiple sessions. Sensation settings that promote an ideal degree of hormesis in the subject are kept for this subject; other such settings that produce a suboptimal degree of hormesis are discarded.

This optimal combination for the subject is stored in one or more of ARC server 151, mobile app 822a, and services 821a.

### Disinfecting the Enclosure

In an embodiment, sauna 110 includes one or more ultraviolet (UV) lamps for disinfecting the interior of the space. With the emergence of SARS-COV-2 in late 2019, the awareness of communicable pathogens in shared spaces has become acute in much of the general public. Moreover, given that scientific studies have shown that heat can improve the health of sick people, the chances that a sick person will use sauna 110 is encouraged and thus increased. This makes it more probable that sauna 110 may well harbor pathogens after certain people make use of the space.

UV light is well known in the art for its ability to disinfect both surfaces and the air of viruses, bacteria, and other pathogens. This disinfection acts to prevent the spread of illness and ensure a clean and safe environment for subjects.

Although sauna 110 could use any wavelength of UV known in the art to disinfect surfaces, an embodiment uses one or more wavelengths between 220 nm and 230 nm to maximize the allowable threshold limit values of UV on the eyes and skin of any person who accidently comes in contact with the UV light.

In an embodiment, one or more UV lamps are embedded within any one or more of the panels of sauna 110. For example, sensor 1001 embedded in rip curl 313 shown in FIG. 10 is a UV lamp. In another example, a UV lamp is embedded in full spectrum heater 124.

In an embodiment, the UV lamp is turned off while a subject is present within sauna 110.

In an embodiment, sauna 110 initiates a disinfection session automatically by turning on the UV lamp once presence sensor 165a and door sensor 167 indicate, respectively, that no subject is present within sauna 110, and door 317b is closed.

This disinfection session operates for a prescribed time—for example, 15 minutes—and sauna 110 turns off the UV lamp once that time is up. In addition, the UV lamp is turned off as soon as presence sensor 165a or door sensor 167 indicate, respectively, that a subject is present within sauna 110, or door 317b is open.

In an embodiment, the subject activates a disinfection session via UI item (e.g. a “Sanitize Device” button) in mobile app 822a.

In an embodiment, the UV lamp is covered by a small panel when not in use. Once a disinfection session begins, an actuator is engaged to move the small panel so that the light from the UV lamp can reach the interior of sauna 110.

In an embodiment, in addition to the UV lamp, or in alternative, sauna 110 includes a UVC fan (such as the UV-Fan sold by Cognituv™) at the air intake opening located in panel 316. This UVC fan cycles sanitized air inside sauna 110.

In contrast with the UV lamp, the UVC fan can operate while a subject in inside sauna 110 a session is in progress, thereby funneling UV-C-treated air into each session. The UVC fan is installed in sauna 110 so that the subject does not come in contact with the fan's UVC light.

### EMF Amelioration

One concern of people who use infrared saunas is the level of electric and magnetic fields (“EMFs”) present within the enclosure. One approach for minimizing EMFs taken by manufacturers of such saunas involves coating each electrical line and component with special-purpose insulation. Such an approach raises the cost of such saunas while failing to eliminate all EMFs in all areas of the enclosure.

Much has been written about the nature of harms from EMF, ranging from heating of tissue to cancer to a range of only partially explained symptoms such as fatigue and headache. There is some thought that connecting the skin of the person (e.g. bottom of bare feet) to electrical ground may help in eliminating the harms of EMF.

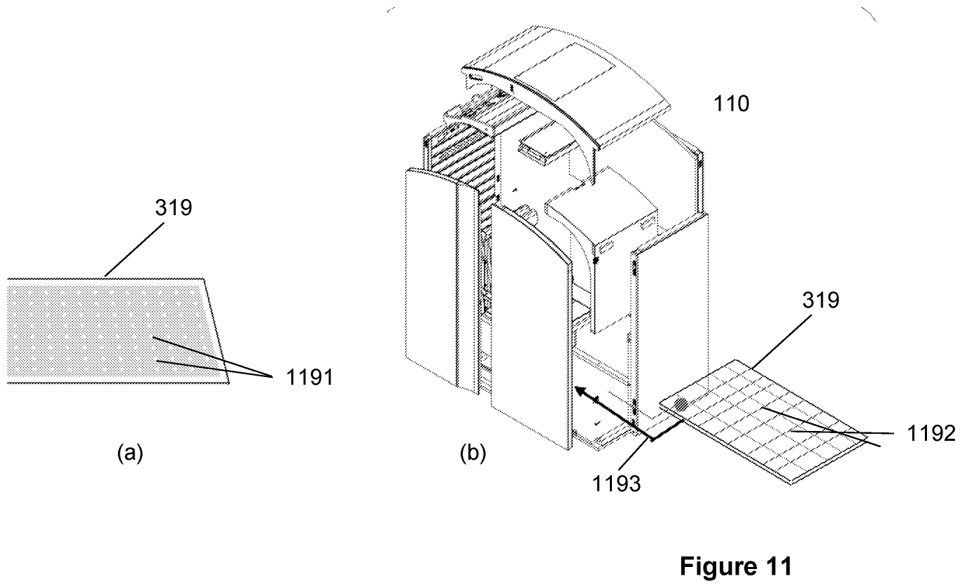

FIG. 11 shows sauna 110 including EMF-ameliorating features, according to an embodiment. In an embodiment of these features, FIG. 11(a) shows metal studs 1191 embedded within and dispersed around floor 319. Each such metal stud 1191 runs from the top of floor 319 to the bottom. When a subject is sitting in chair 416 within sauna 110 with her bare feet resting on floor 319, at least parts of one or both of the bottom of her feet are in contact with one or more of the metal studs 1191.

The metal studs 1191 comprise any type of metal that is relatively high in electrical conductivity, such as copper. In an embodiment, the metal studs 1191 comprise aluminum, copper, silver, gold, graphene, and any combination thereof.

Below floor 319 is a conducting system that electrically connects to all of the bottoms of the metal studs. In an embodiment, this conducting system comprises an electrical mesh 1192 of electrical lines, which connects to the bottoms of one of all of the metal studs 1191. An end of electrical mesh 1192 connects to ground electrical line 1193 that connects all of the metal studs 1191 to ground within power system 170.

In an embodiment, this conducting system comprises a two-dimensional metal plate that covers the bottom of floor 319 and is comprised of the same kind of metal used for the metal studs 1191. At one edge or corner of the metal plate, ground electrical line 1193 connects the metal plate (and thus all of the metal studs 1191) to ground within power system 170.

So long as the subject keeps her bare feet on floor 319 during the session, the subject may be at least somewhat protected from harms of any EMFs present within sauna 110. The subject is informed to remain bare-footed while in sauna 110 by one or more of IUC 161, mobile app 822a, and sound system 130.

In an embodiment, the EMF-ameliorating features comprise a removable grounding mat known in the art. For example, EarthFX, Inc. sells such grounding mats. In this embodiment, ground electrical line 1193 attaches electrically to the grounding mat at one end, and to ground within power system 170 at the other end.

### Detecting Physical Health Improvements

In an embodiment, sauna 110 uses the sweat collection methods discussed above on page 17 (e.g., use of wearable sensors for continuous measuring of cortisol levels in human sweat) to measure other health-related metrics, including one or more of heat shock proteins (HSPs) and metals present in the sweat.

It is known in the art that during heated sessions, the human body is capable of generating HSPs, which is understood to be a healthy reaction. It is also known in the art that HSPs are expressed in the skin (see, e.g., “Cell stress and implications of the heat-shock response in skin,” available at https://pubmed.ncbi.nlm.nih.gov/8564645/, which is incorporated by reference herein). As such, HSP sensors in contact with sweat will soon be able to measure HSPs.

In an embodiment, sauna 110 includes such HSP sensors for sweat as discussed above regarding the cortisol sensors.

It is known in the art that one principal benefit of infrared heated sessions is detoxification of the body of heavy metals. During a session, these metals are expelled by the body via sweat. In an embodiment, sauna 110 employs a heavy metal sensor that uses light to detect heavy metals on human skin (see, e.g., “Wearable Microsensor Array for Multiplexed Heavy Metal Monitoring of Body Fluids”). As with the remote temperature sensor discussed earlier, the remote metal sensor is embedded in a panel of sauna 110 remote from the subject (for example, in rip curl 313 as shown in FIG. 10 as sensor 1001).

The results of the foregoing direct health measurements are presented to the subject via one or more of IUC 161, mobile app 822a, and sound system 130. Across multiple sessions, the subject can track her improving health, shown by increases in HSPs and reductions in heavy metals.

### Social Hour

In an embodiment, ARC services 821a streams live sessions, at certain times and dates, to one or more sauna 110 devices as a social hour. Certain people feel greater motivation to stop their normal hectic activities to engage in a parasympathetic-inducing session knowing that others around the country and the world are doing the same. This motivational effect is amplified by providing a social media web page or mobile app 822a where these subjects can share their experiences before and after the live session.

When a subject has confirmed to join a stream session, ARC services 821a then interfaces with ARC server 151 to stream one or more audio and light data to sauna 110. The audio stream acts similarly to a live performance from a streaming service as known in the art, and the lighting data is a live performance of lighting that is serialized and transmitted similarly to how a video accompanies streaming audio on video platforms as is known in the art.

The content creator is the person who leads the live session. The content creator may access a live streaming platform, a content creator interface, and a viewer interface. The live streaming platform enables the content creator to broadcast a virtual sauna environment, complete with adjustable light and sound settings. The content creator interface provides control options for adjusting these settings in real-time, while the viewer interface allows subjects to interact with the content creator, provide feedback, and access curated light and sound settings.

In an embodiment, the content creator is present at an Augmented Reality (AR) studio, where the audio, lighting, and other environmental settings are directly streamed to multiple saunas 110 that are attending to the live session. Alternatively, the saunas 110 are communicatively coupled via a locally wired connection. The AR studio is equipped with advanced hardware and software known in the art, enabling the content creator to control and adjust the virtual sauna environment in real-time for the subjects.

In an embodiment, the content creator controls the live stream sauna session from their home or from any other location remote from the AR. The leader uses a dedicated dashboard on their computer or a compatible device, allowing them to control the audio, lighting, and other environmental settings. The leader's voice and light settings are streamed to the AR studio, and from there, these are transmitted to the multiple enclosures 110 that are attending the live session. This embodiment eliminates the need for the AR to be a complex physical studio setup, as there is no video component involved in the live stream.

In both embodiments, during a live stream sauna session, the content creator selects and adjusts light and sound settings to create a customized sauna experience for the subjects. Sauna 110 enables real-time adjustments based on viewer feedback (submitted via one or more of microphone 172 or IUC 161) or the content creator's preferences, enhancing the immersive and participatory aspects of the live stream experience.

### Modular Furniture on the Outside of the Enclosure

In an embodiment, sauna 110 can support various fixtures attached to the outside side of one or more of its panels. These fixtures serve to increase the utility of sauna 110 in its setting, for example, a room in a home, or in a public building like a gym. This embodiment makes it more likely that sauna 110 will be installed within frequently visited rooms such as a family or living room, a choice outdoor setting, luxurious location, and more. This choice placement of sauna 110 should promote its usage by subjects.

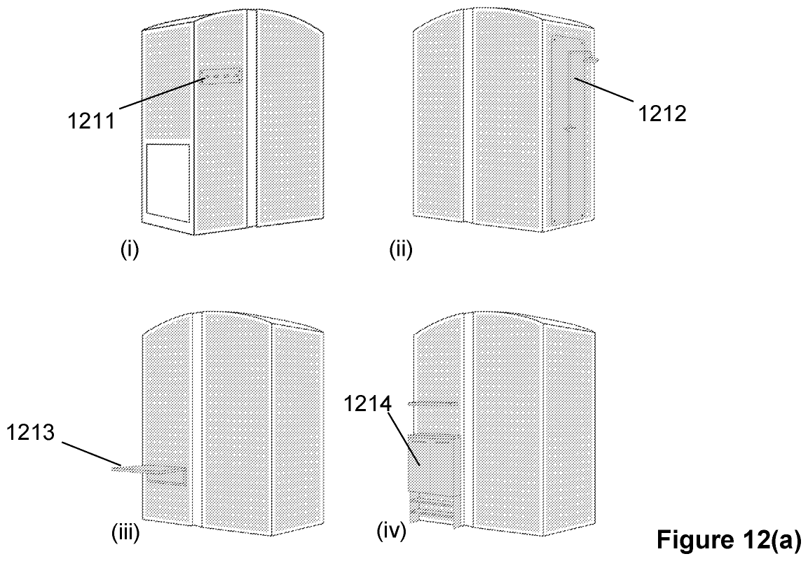

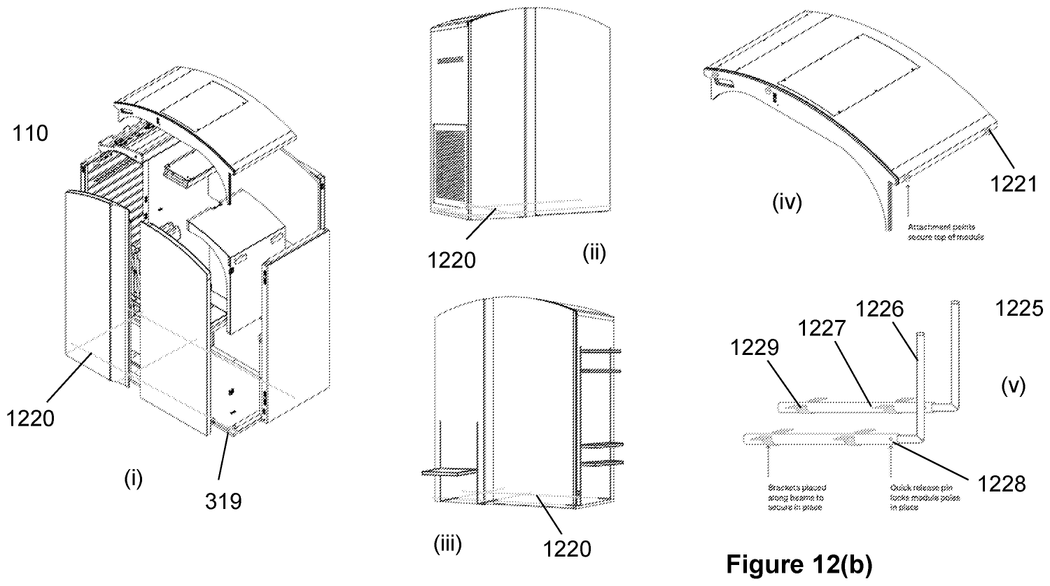

FIG. 12 shows sauna 110 including features for coupling fixtures to the outside of sauna 110, according to an embodiment. FIG. 12(a) shows examples of fixtures attached to sauna 110. In the embodiment shown in FIG. 12(a), these include (i) coat hooks 1211, (ii) a shower 1212, (iii) a seat 1213, and (iv) shelving 1214. Additional compatible fixtures include a table, a bench, a cryotherapy chamber, cold plunge, massage table, living plant wall, storage compartment, and the like. Moreover, while FIG. 12(a) shows these fixtures attached to either right wall 317a or left panel 318, the fixtures are configured to attach to exteriors of any of panels of sauna 110.

FIG. 12(b) shows a strong framework 1220 for supporting the fixtures. Strong framework 1220 is embedded in the one or panels of sauna 110. Although strong framework 1220 is shown in FIG. 12(b)(i), (ii), and (iii) embedded in floor 319, in other embodiments, this framework is embedded in any one or more of the panels of sauna 110. Strong framework 1220 is comprised of any strong material such as steel, graphene-based materials, etc.

FIG. 12(b)(iv) and (v) show an embodiment of how the fixtures are coupled to the panels of sauna 110. FIG. 12(b)(iv) shows a plurality of upper attachment mechanisms 1221 located around the edges of roof 314. Upper attachment mechanism 1221 comprises a durable and secure hook component and is designed to attach fixtures to roof 314.

FIG. 12(b)(v) shows a plurality of bottom supporting mechanisms 1225 located around the edges of floor 319. Each mechanism 1225 comprises a supporting pole 1226, a housing 1227, quick release pin 1228, and brackets 1229. In this embodiment, each fixture includes a vertical oriented cylindrical housing that slips over supporting pole 1226.

Supporting pole 1226 is attached to sauna 110 via housing 1227. Housing 1227 is part of strong framework 1220. When in use, supporting pole 1226 protrudes sufficiently from the sauna 110 panel to enable the housings of the fixture to slip over the poles. When not in use, supporting pole 1226 can be pushed back to the panel.

For moving supporting pole 1226 in and out of housing 1227, this embodiment provides quick release pin 1228. When this pin is pushed inward, supporting pole 1226 is free to move within housing 1227; when fixed in the outside position, quick release pin 1228 serves to lock supporting pole 1226 in its position.

Each hook component of upper attachment mechanism 1221 works in conjunction with a bottom supporting mechanism 1225 to ensure stability and prevent displacement of the fixture. When not in use, a D-ring component of upper attachment mechanism 1221 sits flush with the perimeter of the line of roof 314, creating a sleek and streamlined appearance.

Upper attachment mechanisms 1221 and bottom supporting mechanisms 1225 are comprised of any material strong enough to support heavy fixtures, including stainless steel, graphene-based materials, etc.

### Reclining Chair

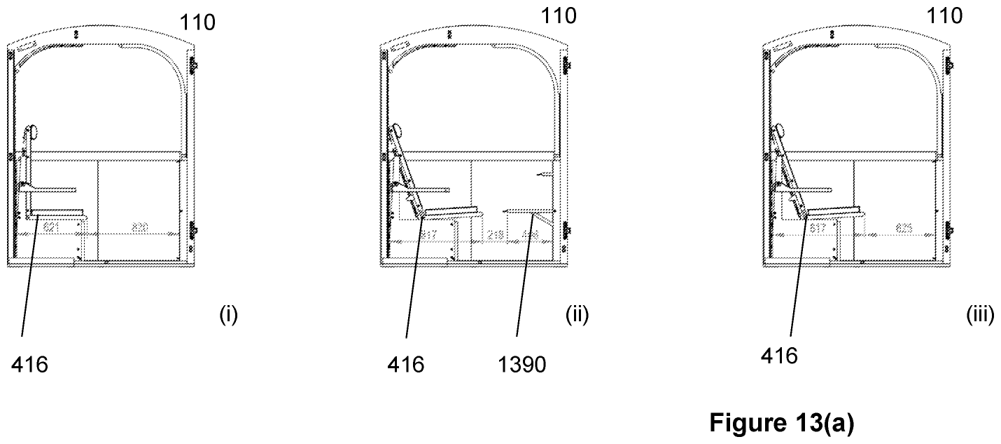

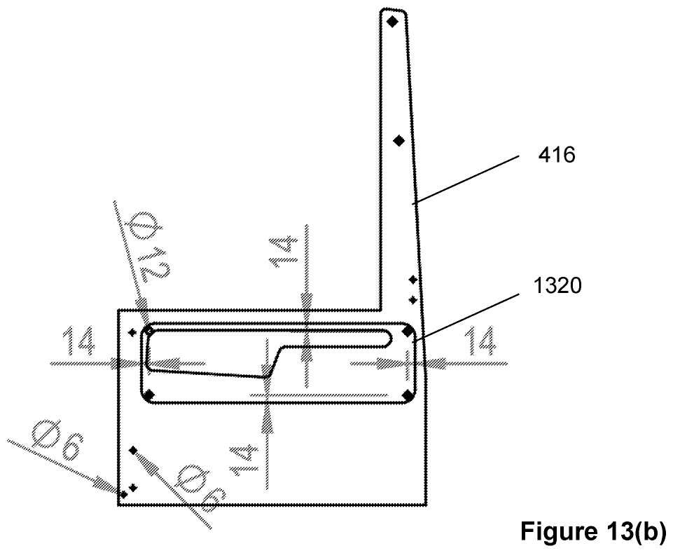

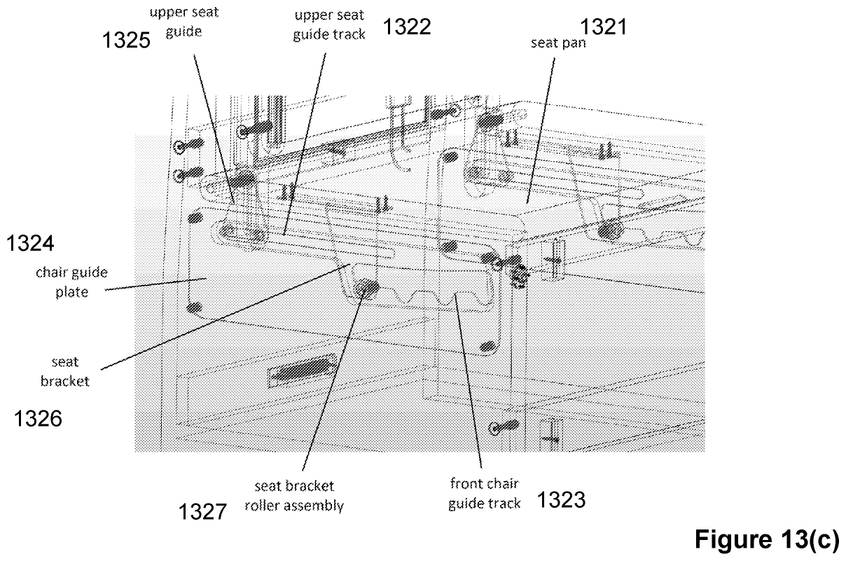

FIG. 13 shows sauna 110 including a reclining chair 416, according to an embodiment.

FIG. 13(a) shows an interior view of sauna 110, according to an embodiment. This interior includes reclining chair 416. In FIG. 13(a)(i), reclining chair 416 is shown in the upright position. In FIG. 13(a)(ii) and (iii), reclining chair 416 is shown in the reclined position.

FIG. 13(a)(ii) also includes bench 1390.

FIG. 13(b) shows a side view of reclining chair 416. Reclining mechanism 1320 is shown embedded within reclining chair 416.

FIG. 13(c) shows an oblique view of reclining mechanism 1320. The subject adjusts the recline of chair 416 by pulling the front of seat pan 1321, lifting upwards and sliding forward to recline, and backwards to return chair 416 to a more upright position. Chair 416 enables a plurality of positions of recline as determined by front chair guide track 1323, which is connected to and supported by chair guide plate 1324. Seat bracket roller assembly 1327 drops into a groove of front chair guide track 1323, which fixes seat 416 in any one of the recline positions.

We claim:

1. A system for inducing a parasympathetic state in an individual wherein the system is comprised of:
a) a portable sauna;
b) multi-sensory transducers;
c) an operating system; and
d) a power system.

2. The system of claim 1, wherein the sauna is comprised of an enclosure for housing at least one individual.

3. The system of claim 1, wherein the system is further comprised of an electrical cabinet having an ARC server and a sensor system.

4. The system of claim 3, wherein the sensor system is comprised of at least an in-sauna controller, a presence sensor, a temperature sensor and a door sensor.

5. The system of claim 1, wherein the sauna further includes a plurality of sensors capable of measuring heart rate, EEG data, respiration, cortisol levels, heat shock proteins and heavy metals.

6. The system of claim 1, wherein the power system is further comprised of a power system converter and a microphone.

7. The system of claim 1, wherein the multi-sensory transducers include a heating system, a sound system, and a lighting system.

8. The system of claim 7, wherein the heating system is further comprised of a heating controller, a full spectrum heater and infrared panels.

9. The system of claim 7, wherein the sound system is further comprised of an audio amplifier, speakers, and a subwoofer.

10. The system of claim 7, wherein the lighting system is further comprised of an LED controller and 16-bit LED bulbs.

11. The system of claim 1, wherein the system is connected to a network for transmittal of data to and from the system.

12. The system of claim 7, wherein the lighting system is further comprised of one or more ultraviolet lamps for disinfecting the interior of the enclosure.

13. The system of claim 1, wherein the sauna is further comprised of a floor that can ameliorate electric and magnetic fields.

14. The system of claim 2, wherein the enclosure further includes a chair having a reclining mechanism.

15. The system of claim 2, wherein the enclosure further includes at least one bench.
